(19) United States
(12) Patent Application Publication
(10) Pub. No.: US 2026/0209821 A1
(43) Pub. Date: Jul. 23, 2026

(54) METHODS, SYSTEMS, AND TEST KITS FOR ASSESSING ANTI-MICROBIAL VISCOUS LIQUID

(71) Applicant: SWIMC LLC, Cleveland, OH (US)

(72) Inventors: Tony A. ROOK, Cleveland, OH (US); Michael C. LAMBERTSON, JR., Cleveland, OH (US); Carl BRAUN, Cleveland, OH (US); Kimberly PERCIVAL, Cleveland, OH (US)

(21) Appl. No.: 19/139,097

(22) PCT Filed: Dec. 15, 2023
(86) PCT No.: PCT/US2023/084182

§ 371 Date: Jun. 13, 2025

Related U.S. Application Data
(60) Provisional application No. 63/433,207, filed on Dec. 16, 2022.

(51) Int. Cl. C12Q 1/18; C09D 5/14; C12Q 1/24; C12Q 1/70

(52) U.S. Cl. CPC C12Q 1/18; C09D 5/14; C12Q 1/24; C12Q 1/70

(57) ABSTRACT

Methods, systems, apparatuses, and kits are disclosed to evaluate or test the antimicrobial and microbicidal characteristics and properties of viscous liquids such as paints and coatings applied by contact means such as smearing, brushing, or drawdown.

### BACKGROUND

Many types of viscous liquids are formulated to have microbicidal characteristics. A procedure and method for evaluating and testing the microbicidal characteristics and properties of material that are tested or applied in their liquid state, such as coatings and paints, has been reported. This procedure and method, for example, is included in Protocol MRID 48482503 and Protocol MRID 50025401. This method involves applying a selected microorganism to the surface of a carrier and then spraying a substance on the carrier. This method is limited to the use of a spraying application, however, and does not encompass other, common means of applying a viscous liquid to a substrate, like brushing, draw down, or rolling. Consequently, it also does not encompass all formulations because some are developed specifically for application by non-spray means. In contrast to spray application, these other application means require contact between the applicator used to apply the viscous liquid, and the test bed containing microbes. Existing methods to evaluate the microbicidal characteristics of compositions applied by spray cannot be used because the microbes that are involved in the test tend to be spread, resulting in faulty and inconsistent data.

Thus, there is a need for a method and means to evaluate and test viscous liquid substances for microbicidal characteristics by direct application means that will produce reliable and consistent data.

### SUMMARY OF THE DISCLOSURE

This disclosure describes processes and procedures to evaluate or test viscous liquids. Disclosed embodiments can determine the antimicrobial and microbicidal characteristics of different types of materials including, but not limited to, oils, mineral slurries, foods, jellies, latex polymers, personal care cream products, nail polishes, medicinals, medicaments, paints, coatings, and precursors therefor.

In one embodiment, this disclosure describes a method for assessing the microbicidal characteristics of a viscous liquid material or substance. This method may comprise the steps of a) forming a microbial film on one or more carriers; b) exposing the microbially-treated carriers to a uniform film of a viscous liquid using a platform containing one or more wells; c) lifting the film-coated carriers from the one or more wells using a tool to lift the carriers out of the wells; d) removing the film-coated carriers from the platform using a tool to move the carriers from the platform; e) incubating the film-coated carriers in media; and f) determining the viability of microbes on the microbial-treated carriers to assess the microbicidal characteristic of the viscous liquid. In alternative embodiments, a viscous liquid uniform film is formed by smearing, rolling, brushing, or drawing down the viscous liquid on the microbial-treated carrier. Those skilled in the art will understand that that the microbicidal properties of the liquid against various microbes may be evaluated using the described method, the microbes including but not limited to, a fungal spore, a mold, a bacteria, a virus, or selected combinations of these microbes. Those skilled in the art also will understand that the microbicidal properties of the liquid against different cells may be evaluated using the described method, such as but not limited to, skin cells, blood cells, cells from tissue biopsies. a cultured cell sample, or a combination of such cells.

This disclosure describes a method for depositing a thin film of a liquid for testing on a treated carrier comprising the steps of forming a microbial film or material film on one or more carriers; exposing the carriers treated with the microbial film or material film to a thin film of a liquid using a platform containing one or more wells, the thin film deposited by drawing down the liquid on the platform; lifting the film-coated carriers from the wells using a tool to lift the carriers from the multiple wells; and removing the film-coated carriers from the platform using a tool to move the carriers from the platform. In one embodiment, the uniform film of the viscous liquid is prepared by a drawdown process using a drawdown bar to uniformly apply a thin film of a liquid, such as, for example, a paint or coating, on the microbial-treated carrier. However, the liquid film also may be deposited by smearing or brushing the liquid film. In contrast to prior methods, the present disclosure allows for assessment of liquids that require contact between the applicator and the carrier. Whereas prior methods did not support assessment of liquids applied in such ways because contact would smear the microbial or material film present on the carrier, the present methods supports application by such means and can differentiate between liquid formulas that provide more or less microbicidal activity against the microbial film or material film present on the carrier.

The present disclosure also describes a system to prepare uniform films on carriers for further evaluation, processing or testing. In some embodiments, the system may comprise a well platform containing wells to hold the carrier, a lifting tool to lift a microbial-coated carrier from a well in the well platform, and a tool to move a carrier from the well platform. The lifting tool may be configured to comprise a lifting structure, such as, for example, tabs, fingers, protrusions, or prongs, that will match with the features of the multi-well platform to remove the carrier from the wells of the well platform.

Other embodiments described in this disclosure comprise a test kit for assessing the resistance of a paint or coating to mold or fungal growth. Features of the test kit may comprise carriers, a well platform, a drawdown bar, a lifting tool to lift a microbial-coated carrier from a well in the well platform, and a removal tool to move the microbial-coated carriers from the well platform as well as suitable vessels and tools for preparing and handling microbial-treated carriers. Additional optional features of the kit may comprise microbes, growth media reagents, and neutralization reagents.

The foregoing embodiments may be combined with one or more optional features as further described herein.

### DESCRIPTION OF THE DRAWINGS

FIG. 1 is an isometric view of a multi-well platform.

FIG. 2 is an isometric view of a lifting tool.

FIG. 3 is an isometric view of a removal tool.

FIG. 4 is a top view illustrating a film-coated carrier being lifted from a multi-well tool by the lifting tool in order to allow the removal tool to transfer the film-coated carrier for further processing.

FIG. 5 is a side view of FIG. 4 illustrating a carrier film being lifted from a multi-well tool.

### DETAILED DESCRIPTION

This disclosure describes processes and procedures to evaluate or test the antimicrobial and microbicidal characteristics and properties of viscous liquids. Disclosed embodiments provide devices, tools and methods providing consistent and reproducible results to prepare samples coated with liquid substances and to handle the samples in subsequent test processes.

In this disclosure, specific embodiments have been described above with reference to the disclosed embodiments and examples, such embodiments are only illustrative and do not limit the scope of the disclosure.

All publications, patents, and patent documents are incorporated by reference herein, as though individually incorporated by reference. No limitations inconsistent with this disclosure are to be understood from such publications, patents, and patent documents.

### Methods

This disclosure describes contacting a microbially-treated carrier or substrate with a liquid for a predetermined time, the liquid applied by contacting the carrier with an applicator soaked in the liquid, removing the carrier via aseptic techniques, and then assessing the viability of microbes on the carrier surface to grow under conditions that provide for quantifiable microbial activity. Also disclosed are a testing apparatus and kit for performing the disclosed methods. The disclosed methods thus allow for quantification of the microbicidal characteristics of the viscous liquid.

In some embodiments, selected isolated microbes are applied to suitable carriers, such as glass slides, according to established protocols known to those skilled in the present fields of evaluation and testing microbial characteristics. Carriers are sterilized prior to application of the isolated microbes use to limit or eliminate contamination by outside microbes. Carriers may be configured in a variety of shapes and sizes but preferably are thin glass slides. In addition, carriers may be made from a variety of materials, such as glass, metal, plastics or polymers and preferably are non-porous. Carrier materials may be selected to be specific for the microbes used in the evaluation or testing. Suitable carrier materials will support microbial film formation, allow for processes using growth media reagents, neutralize agents, or other reagents, and will be used to provide for qualitative or quantitative determination of the microbiocidal effect of the liquids being evaluated or tested according to this disclosure.

The microbes used may broadly include one or more microbial or cellular-based samples comprising, for example, bacteria, viruses, or fungi, as well as isolate or treated animal cells. Suitable animal cells include, for example, skin cells, blood cells, or cells from tissue biopsies. In addition, a cell sample may be a cultured cell sample. A cultured cell sample may be used when a cell or small group of cells is expanded using known culture processes. When the cell culture reaches a desired cell density, it may then be further processed, if needed, and then applied to a suitable carrier forming a microbe-based film.

The viscous liquid is applied to the microbially covered slides by means that require contact between the viscous liquid, such as a coating, and applicator, such as brush, roller, or drawdown. Unlike prior methods, which supported testing viscous liquids applied only by spray, the present disclosure provides specific support for application by contact means, which ordinarily would result in undesirable smearing or spreading of microbes on the carriers. The present disclosure also allows for control of the amount of viscous liquid applied on each carrier, thus, allowing a volumetric assessment of microbicidity on the volume basis.

Thus, in embodiments of the presently described method, an accurate amount of viscous liquid can be dispensed in a small area without compromising the integrity of microbes present on the carrier surface. The viscous liquid may be an oil, mineral slurry, foodstuff, jelly, latex polymer, personal care cream product (e.g., cream, lotion), nail polishes, medicinal (e.g., athletes foot creams), or any other viscous liquid that may be drawn down on a test carrier.

Suitable microbially-treated sample test carriers are placed in a well platform having independent wells for the carriers (the well platform can also be referred to as a drawdown template), and the viscous liquid is applied to the carriers by drawing down using a straight edge, brushing, or by roller. This allows a viscous liquid, such as a paint or coating, to be applied on top of microbe-covered slides using usual viscous liquid application techniques which require contact between the viscous liquid and applicator, without removing the microbes from the carrier surface by friction or fluid flow. The film-coated carriers in the template wells may then be placed over a lifting tool to help lift the film-coated test carriers off of the drawdown template well. A slide remover is then used to lift the sample test carriers off of the lifting tool without direct human contact with the carriers, and transfer the samples into a safe place to continue with a test to assess the viability of microbes present on the carrier to grow. The use of the drawdown template in this method provides consistent film build up over the multiple carriers in the wells of the drawdown template without compromising the microbes present on the surface of the carriers.

After the uniform film is applied on the carrier, the carrier is lifted from the well platform and transferred to a suitable growth or nutrient broth for further processing. A suitable nutrient broth for the microbe being used in the evaluation or testing method is selected. In some approaches, the nutrient broth may be sabouraud dextrose media, potato dextrose media, malt media, glucose media, minimum salts media, trypticase soy media, nutrient media, brain heart infusion media, plate count media, R2A media, blood media, or charcoal media, or another suitable nutrient media. While in the nutrient broth, the film-coated carrier may be kept at a desired temperature allow expansion or growth of the transferred microbes. It is well known that there are microbes which will grow best at elevated temperatures, such as, for, example 37° C. In some embodiments, the incubated film-coated carriers are agitated at elevated temperatures for a predetermined period of time in a range of several hours to several days.

When the period of incubation time ends, the results of the evaluation or testing can be determined using many types of qualitative or quantitative processes. In some embodiments, the incubated sample is contacted with a neutralizing reagent solution and the turbidity of the solution can be visually analyzed for the microbiocidal effect of the viscous liquid being evaluated or tested. The neutralizing agent may, for instance, neutralizers and inactivators of ASTM E1054-08 (2013), titled Standard Test Methods for Evaluation of Inactivators of Antimicrobial Agents. Alternatively, aliquots from the nutrient broth may be added to growth media agar in a Petri dish or an agar coated film such as a PETRIFILM count plate (commercially available from 3M, Saint Paul, MN) and microbial viability can be determined using established methods.

In some embodiments, immediately after contact time of the viscous liquid with the treated carrier, the carrier is removed from the test apparatus using a lifting tool and is added to a sterile primary testing vessel. Sterile lecithin/dextrose based neutralizing broth media (e.g., 20 mL) is added to a primary vessel and the carrier is dropped into the broth media. Any test substance remaining on the carrier may be scraped off using a sterile cell scraper in the primary neutralizing vessel. The carrier is then placed into a secondary vessel containing unused neutralizing media (e.g., 20 mL) using the cell scraper as a spatula to transfer the carrier.

All primary and secondary vessels are then incubated at a predetermined time necessary to assess microbial growth. In some approaches, this may require incubation, for instance, at 30° C.+/−3° C. for 10 days. After the incubation period, 200 μL aliquots are taken from each of the primary vessels and plated on suitable agar and incubated for 5 days at 30° C.+/−3° C. Additional spread plates may be used to confirm if fungal growth observed in the primary and secondary vessels have characteristic colors of the microbe colonies being tested.

The criteria for a pass result may be zero recovery of test organism from primary neutralization vessels, secondary neutralization vessels or any spread plates taken from primary and/or secondary vessels from all replicates. In some approaches, the criteria for a pass result may be adjusted, such that a pass result is a low number of test organism recovered from the primary neutralization vessel or a secondary neutralization vessel. In this way, the criteria can be adjusted to allow for comparative testing between samples.

In a selected embodiment, this disclosure describes a method for assessing the resistance of a paint or coating to mold or fungal growth comprising preparing microbial-treated carriers; placing the microbial-treated carriers into a platform containing one or more wells; forming a uniform film of the paint or coating sample on microbial-treated carriers using the platform containing wells and a drawdown bar to provide film-coated carriers; exposing the carriers to the paint or coating samples; lifting the film-coated carriers from the wells using a tool to lift the carriers out of the multiple wells; removing the film-coated carriers from the one or more wells using a tool to move the carriers from the platform; incubating the film-coated carriers in media for a predetermined incubation time at a temperature greater than ambient temperature; neutralizing the media with a neutralizing reagent solution; and determining the viability of microbes in the neutralization reagent solution to assess the resistance of the paint or coating to mold or fungal growth by a qualitative macroscopic measurement of the turbidity of the neutralizing solution and quantitative assessment of mold or fungal growth on a spread plate of the neutralizing solution.

### Platform and Tools

Suitable preparation and handling devices and tools that can be used in the disclosed methods include single-well or multi-well platforms, drawdown tools, lifting tools and transfer tools. Various features of these devices and tools are described below in sections describing these devices and tools in detail.

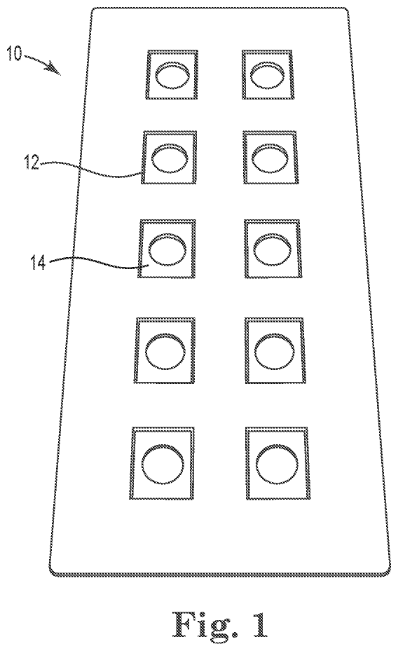

FIG. 1 is an embodiment of a multi-well platform or tool. In this embodiment, the multi-well platform 10 comprises ten wells 12 holding ten rectangular carriers 14. These wells are sized to hold the carriers containing selected microbes. The wells have a depth that is greater than the thickness of the carriers. The depth of the well is selected to allow a film of desired depth of the viscous liquid substance to from on the exposed upper surface of the carriers. As part of the procedure, the viscous liquid substance is spread across the carrier using a drawdown application. However, in some embodiments, the viscous liquid may be spread by smearing, brushing, or another contact application. In embodiments using application by drawdown, the viscous liquid substance is contacted with the upper surface of the well platform and a sanitized straight edge tool is drawn horizontally over the wells to form a uniform liquid film in each well. Further, each well has an aperture or opening to provide contact with a carrier lifting tool. The number of wells may be one, two, three, four, or more. The shape of the wells, or the depth of the wells can, for example, be selected from a variety of numbers, shapes or depths in order to optimize these features for the type of microbes to be evaluated or tested as well as the characteristics and properties of the viscous liquid providing the film-coated carriers.

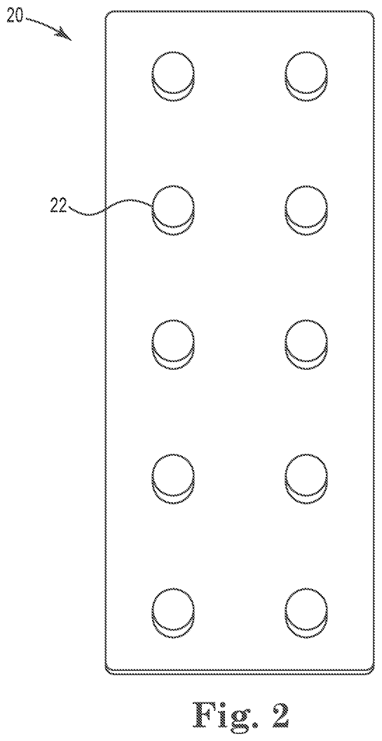

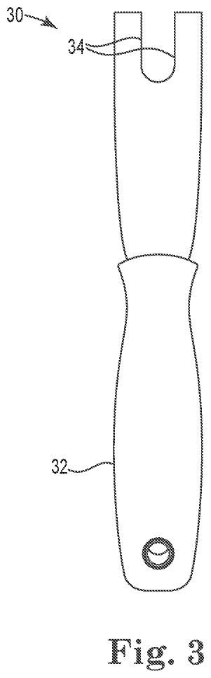

FIG. 2 is an embodiment of a carrier lifting tool 20. The carrier lifting tool comprises structure 22 extending vertically from a surface of the carrier lifting tool. Suitable structures may include, for example, tabs, fingers, protrusions, or prongs. These structures fit into and through the apertures or openings in each well and have a vertical height allowing the lower surface of the carrier to be lifted from the well above the upper surface of the multi-well platform. In an embodiment, a carrier removal tool, illustrated in FIG. 3, is inserted between the upper surface of the multi-well platform and the bottom surface of the carrier to remove the carrier for subsequent processing during the evaluation or testing procedure.

FIG. 3 is an embodiment of a carrier removal tool 30. The carrier removal tool comprises features to remove the carrier from the multi-well platform. A handle 32 on the removal tool provides for contacting and removing the carrier in a sanitary procedure avoiding undesired contamination of the carrier. Lifting prongs 34 on the removal tool provide a structure to contact and lift the carrier when suspended on the lifting structures of the carrier lifting tool above the upper surface of the multi-well platform. The prongs fit around the outside of the lifting structures to allow a stable procedure to remove and transport the carrier. In some aspects, the lifting prongs may include an aperture for receiving the carrier.

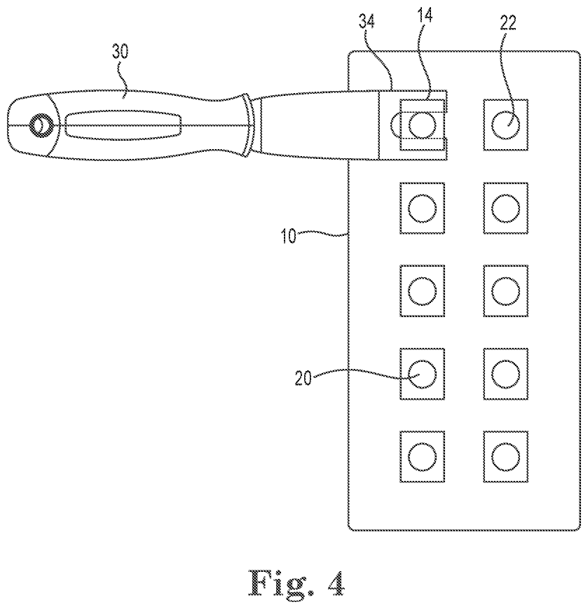

FIG. 4 is an embodiment of one step of the evaluation or testing processes or procedures of this disclosure. The illustrated step of FIG. 4 is an embodiment with the vertical structures of the carrier lifting tool 20 fitted into and extended through the apertures of the well platform 10 suspending the carrier 14 above the upper surface of the multi-well platform. The carrier removal tool 30 is fitted between the upper surface of the well platform and the lower surface of the carrier and the prongs 34 of the carrier removal tool are fitting around the outside of the lifting structures. The two prongs of the carrier removal tool will allow the carrier to be readily removed and transported in subsequent procedures.

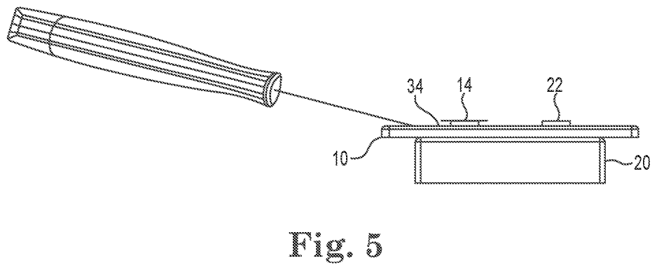

FIG. 5 is a side view of the procedural step illustrated in FIG. 4. This side view illustrates the positions of the components when the carrier 14 is suspended above the upper surface of the well platform 10. In this figure the structures 22 of the lifting tool 20 are fitted into and extended though the apertures of the multi-well platform suspending the carrier above the upper surface of the well platform. Further, the prongs 34 of the carrier removal tool fit into the area between the upper surface of the well platform and the bottom surface of the carrier.

The well platform, lifting tool and removal tool may be made from any materials that can be sterilized. Suitable materials include, for example, glass, metal, or plastics. Combinations of suitable materials may also be used. In addition, he well platform, lifting tool and removal tool may be single-use, disposal materials. Alternatively, the multi-well platform, lifting tool and removal tool may be reusable as long as the devices are cleaned and sterilized before each use.

### Kits and Systems

Features of a testing kit or testing system may comprise components such as carriers, a multi-well platform, a drawdown bar, a lifting tool to lift a microbial-coated carrier from a well in the well platform, and a removal tool to move the film-coated carriers from the well platform as well as suitable vessels and tools for preparing and handling microbial-treated carriers. Additional features of the kit may comprise microbes, growth media reagents, and neutralization reagents. The combination of all the components in kit form makes the method easy to use and avoids unwanted variability of the evaluation or testing when the components for the method are sourced from multiple suppliers or vendors.

As used in this specification and the appended claims, the singular forms “a,” “an,” and “the,” include plural referents unless expressly and unequivocally limited to one referent. Thus, for example, reference to “an additive” includes two or more different additives. As used herein, the term “include” and its grammatical variants are intended to be non-limiting, such that recitation of items in a list is not to the exclusion of other like items that can be substituted or added to the listed items

The recitation of numeral ranges by endpoints includes all numbers subsumed within that range (e.g., 1 to 5 includes 1, 1.5, 2, 2.75, 3, 3.80, 4, 5, etc.). Furthermore, disclosure of a range intended to be a specific disclosure of all subranges included within the broader range (e.g., 1 to 5 discloses 1 to 4, 1.5 to 4.5, 1 to 2, etc.).

The terms “preferred” and “preferably” refer to embodiments of the present invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the present invention.

The term “comprises” and variations thereof do not have a limiting meaning where these terms appear in the description and claim

It is to be understood that each component, compound, substituent or parameter disclosed herein is to be interpreted as being disclosed for use alone or in combination with one or more of each and every other component, compound, substituent or parameter disclosed herein.

It is further understood that each range disclosed herein is to be interpreted as a disclosure of each specific value within the disclosed range that has the same number of significant digits. Thus, for example, a range from 1 to 4 is to be interpreted as an express disclosure of the values 1, 2, 3 and 4 as well as any range of such values.

Those skilled in this field will understand that the description of exemplary embodiments in this disclosure is not intended to limit the disclosure to the particular forms disclosed and that the intention is to cover all modifications, equivalents and alternatives falling within the spirit and scope of the disclosure as defined by the embodiments and the claims below. Reference should be made to the embodiments and claims for interpreting the scope of the disclosure.

The following Examples are intended to illustrate the above disclosure and should not be construed as to narrow its scope. One skilled in the art will recognize that the Examples suggest many other ways in which the disclosure could be practiced. Numerous variations and modifications may be made while remaining within the scope of the disclosure.

### EXAMPLES

The following examples describe a test method for assessing the microbicidity of a test liquid, in this embodiment, a wet-state coating composition. Briefly, a dried film of fungal cells and carrier on the surface of a glass carrier is exposed to a test substance applied by drawdown or spray for 10 minutes exposure time. After exposure, the carriers are transferred to vessels containing neutralizing culture media and assayed for survivors by growth of test organism. The Examples demonstrate the test is capable of differentiating between the microbicidal properties of coating compositions applied by spray or by direct contact applications such as drawdown.

### Example 1

Inoculum preparation: Trichophyton interdigitale (previously known as Trichophyton mentagrophytes), ATCC 9533 obtained from Microbiologics (VWR P/N 89504-368) was grown on Sabouraud Dextrose Agar (SDA, VWR P/N 90000-038) for 12 days at 25° C.

| | Growth | Incubation | |
|---|---|---|---|
| Test Organism | Media | Parameters | Population |
| Trichophyton | Sabouraud | 30° C., | 4.0 × 10\hat{ }7 |
| interdigitale (prev. | Dextrose | aerobic | CFU/mL |
| T. mentagrophytes), | Agar | | |
| ATCC#9533 | | | |

The fungal growth was harvested by scraping from agar into a 50 mL sterile centrifuge tube (VWR #93000-032) containing 8 sterile 3 mm glass beads (VWR P/N 26396-508) and 20 mL of a solution of 0.85% saline and 0.05% Triton X-100 (VWR P/N EM-TX1568-1).

The fungal growth was vortexed at highest speed for 5 minutes then filtered through a sterile Falcon 40 μm pore size cell strainer (VWR P/N 21008-949) into a second sterile 50 mL centrifuge tube.

The fungal filtrate was serially diluted by 1:10 dilutions in sterile DI water then 100 μL aliquots were plated on SDA plates to determine the initial population then adjusted to 4.0×10⁷ CFU/mL in sterile DI water. The adjusted inoculum was kept refrigerated at 4° C.-10° C. until used for testing.

### Sample Preparation

Glass carriers used were 26 mm long×22 mm wide×0.5 mm thick glass hemocytometer slides (Muhwa Scientific P/N MH-800G1). They were prepared by cleaning with 95% ethanol, rinsing with DI water, allowed to dry, then autoclaved for 15 minutes using a 121° C. dry item cycle to sterilize.

Sterile glass slides were placed on an internally produced plastic frame sanitized by immersing in 10% bleach, rinsed with water and allowed to dry.

The adjusted inoculum was mixed to resuspend fungal components.

A 10 μL aliquot of the fungal inoculum was pipetted on middle of the glass carrier and spread with the pipet tip to cover the surface without going over the edge of the glass carrier. Inoculated glass carriers were allowed to dry for 30 minutes in a 35° C. incubator.

After 30 minutes dry time, inoculated carriers were coated with test substance (primer).

After being coated, carriers were removed from the test frame and placed on the inner (unlined) lid (VWR P/N 16087-264) of a sterile 125 mL vessel (VWR P/N 89045-874) with a 60 mm opening. To minimize contamination, an empty vessel was placed over the carrier on the lid for the exposure time at room temperature.

Each inoculated carrier was exposed to test substance for 10 minutes contact time before being recovered. Two different coating application methods were evaluated in separate rounds of testing.

### Drawdown Application

4 mL of coating test sample was pulled horizontally over a carrier in a frame well with a sanitized straight edge, where the depth of the well was the thickness of glass carrier plus 4 mils film thickness of test substance, resulting in a coating thickness of 4 mils.

### Spray Application

Test substance sprayed vertically (approximately 60° angle) with a Wagner Spraytech 0417005 HVLP Control Spray Stain Sprayer until all carrier surfaces covered, typically 1-4 passes required, depending on viscosity of test substance. Sprayer cleaned per manufacturer instructions and allowed to dry between samples.

### Bioburden Recovery

Immediately after 10 minutes of contact time, 20 mL of sterile neutralizing broth media¹ was pipetted into vessel and carrier was dropped into media from lid. Lid was secured onto vessel then vessel was gently shaken for 5 seconds. ¹ Neutralizer broth media was made by combining 0.7 g/L Lecithin (VWR P/N AA36486-30) with 5 g/L Polysorbate 80 (VWR P/N BDH7781-2) in 40% of DI water volume of total volume of media being made and boiling until dissolved. After Lecithin was dissolved completely, 1 g/L sodium thiosulphate (VWR P/N 97061-554), 10 g/L Neopeptone (Fisher P/N 50-201-5951), 20 g/L Dextrose (VWR P/N 18602117) and remaining 60% DI water was added and brought to boiling while mixing with magnetic stir bar. Media was then sterilized in 400 ml bottles using a liquid steam sterilization cycle in the autoclave.

Test substance remaining on glass carrier was scraped off using a sterile cell scraper (VWR P/N 76036-006) in the primary neutralizing vessel. Any growth is recorded.

The glass carrier was then placed into a secondary neutralizing vessel containing 20 mL of neutralizing media using the cell scraper as a spatula to transfer the carrier. Any growth is recorded.

All recovery vessels were incubated at 30° C.+/−3° C. for 10 days.

A 200 μL aliquot was taken from each of the primary vessel at 7 days and plated on SDA2 then incubated for 5 days at 30° C.+/−3° C. to evaluate for test microbe. Additional SDA plates were used to confirm if fungal growth observed in recovery vessel were characteristic white colonies with light orange base of the test microbe.

Criteria for pass was zero recovery of test organism from primary neutralization vessels, secondary neutralization vessels or any spread plates taken from primary and/or secondary vessels from all replicates.

Summary Results for the paint samples tested according to the method of the present disclosure, with the viscous test liquid applied by drawdown and by spray, are listed in Table 1.

TABLE 1
Samples Tested with Summarized Results

| | | Pass/Fail | Pass/Fail |
|---|---|---|---|
| | | (T. interdigitale) | (T. interdigitale) |
| Sample | Description | Drawdown | Spray |
| 1 | Sherwin-Williams Multi-Purpose Interior/Exterior | Pass | Fail |
| | Latex Primer/Sealer | | |
| 2 | Valspar Paint and Primer in one | Fail | Fail |
| 3 | Extreme Block Int/Ext. Primer | Fail | Pass* |
| 4 | Sherwin-Williams PVA Drywall Primer & Sealer | Fail | Fail |
| 5 | Sherwin-Williams Extreme Bond Primer | Pass | Pass* |
| 6 | Foster Products Full Defense 40-20 Fungicidal | Pass* | Pass* |
| | Coating | | |
| 7 | Zinsser Mold Killing Primer | Fail | Pass* |
| 8 | Kilz Mold + Mildew Primer | Pass | Pass* |
| 9 | Fiberlock Aftershock | Pass | Pass* |
| 10 | Sherwin-Williams Paintshield Interior Latex | Pass | Pass* |
| | Paint, Eggshell | | |
| 11 | Sherwin-Williams Loxon XP, Flat Extra White | Pass | Pass* |
| 12 | Sherwin-Williams Duration Exterior Paint, Flat, | Pass | Pass* |
| | Extra White | | |
| 13 | Sherwin-Williams Resilience Exterior Paint, | Pass | Pass* |
| | Satin, Extra White | | |

² Glucose Agar mentioned in the FOIA method is SDA with Emmons modifications (½ dextrose and pH 6.9 vs 5.3). In the example, SDA with pH 5.3 to minimize bacterial growth was used.
*Growth of organism other than test organism (Trichophyton interdigitale, previously Trichophyton mentagrophytes)

More detailed results for samples applied by spray according to method of Example 1 are shown in Table 2.

TABLE 2
Detailed Results for Samples Applied by Spray and Evaluated by Present Method

| | | | Growth in 1st | Growth in 2nd | Growth in |
|---|---|---|---|---|---|
| | | Replicate | Neopeptone | Neopeptone | CFU on |
| Sample | | ID | Neutralizing | Neutralizing | SDA Plate |
| Number | Sample Description | Number | Broth | Broth | (200 uL) |
| 1 | Sherwin-Williams | 1t | None | None | None* |
| | Multi-Purpose Interior/Exterior | 2t | None | None | None |
| | Latex Primer/Sealer | 3t | None | None | None* |
| | | 4t | None | None | None* |
| | | 5t | None | None | 1 CFU |
| 2 | Valspar Paint and Primer in one | 1t | None | None | None* |
| | | 2t | None | None | None* |
| | | 3t | None | None* | None* |
| | | 4t | None | None | None* |
| | | 5t | None | None | None |
| 3 | Sherwin-Williams Extreme | 1t | None | None | None* |
| | Block Interior/Exterior | 2t | None | None | None* |
| | StainBlocking Primer | 3t | None | None | None* |
| | | 4t | None | None | None* |
| | | 5t | None | None | None* |
| 4 | Sherwin-Williams PVA Drywall | 1t | None | None | None* |
| | Primer & Sealer | 2t | None | None | None* |
| | | 3t | None | None | 1 CFU* |
| | | 4t | None | None | None* |
| | | 5t | None | None | None* |
| 5 | Sherwin-Williams Extreme | 1t | None | None | None* |
| | Bond Primer | 2t | None* | None | None* |
| | | 3t | None* | None | None* |
| | | 4t | None | None | None* |
| | | 5t | None | None | None* |
| 6 | Foster Products Full Defense | 1t | None | None | None* |
| | 40-20 Fungicidal Coating | 2t | None | None | None |
| | | 3t | None | None | None |
| | | 4t | None | None | None* |
| | | 5t | None | None | None* |
| 7 | Zinsser Mold Killing Primer | 1t | None | None | None* |
| | | 2t | None | None | None* |
| | | 3t | None | None | None* |
| | | 4t | None | None | None* |
| | | 5t | None | None | None* |
| 8 | Kilz Mold + Mildew Primer | 1t | None | None | None* |
| | | 2t | None | None | None* |
| | | 3t | None | None | None* |
| | | 4t | None | None | None* |
| | | 5t | None | None | None* |
| 9 | Fiberlock Technologies | 1t | None | None | None* |
| | Aftershock Fungicidal Coating | 2t | None | None | None* |
| | | 3t | None | None | None* |
| | | 4t | None | None | None* |
| | | 5t | None | None | None* |
| 10 | Sherwin-Williams Paintshield | 1t | None | None | None* |
| | Interior Latex Paint, Eggshell | 2t | None | None | None* |
| | | 3t | None | None | None* |
| | | 4t | None | None | None* |
| | | 5t | None | None | None* |
| 11 | Sherwin-Williams Loxon XP, | 1t | None | None | None* |
| | Flat Extra White | 2t | None | None | None |
| | | 3t | None | None | None |
| | | 4t | None | None | None |
| | | 5t | None | None | None |
| 12 | Sherwin-Williams Duration | 1t | None | None | None* |
| | Exterior Paint, Flat, Extra | 2t | None | None | None |
| | White | 3t | None | None | None |
| | | 4t | None | None | None |
| | | 5t | None | None | None |
| 13 | Sherwin-Williams Resilience | 1t | None | None* | None* |
| | Exterior Paint, Satin, Extra | 2t | None | None | None* |
| | White | 3t | None | None | None* |
| | | 4t | None | None | None |
| | | 5t | None | None | None |

*Indicates recovery of organism other than test organism (Trichophyton interdigitale, previously Trichophyton mentagrophytes, ATCC 9533).

More detailed results for samples applied by drawdown and evaluated according to the method of the present disclosure are shown in Table 3.

TABLE 3
Detailed Results for Samples Applied by Drawdown and Evaluated by Present
Method Drawdown Application and Evaluated by Present Method.

| | | | Growth in 1st | Growth in 2nd | Growth in | |
|---|---|---|---|---|---|---|
| | | Replicate | Neopeptone | Neopeptone | CFU on | |
| Sample | | ID | Neutralizing | Neutralizing | SDA Plate | |
| Number | Sample Description | Number | Broth | Broth | (200 uL) | |
| 1 | Sherwin-Williams | 1t | None | None | None | |
| | Multi-Purpose Interior/Exterior | 2t | None | None | None | |
| | Latex Primer/Sealer | 3t | None | None | None | |
| | | 4t | None | None | None | |
| | | 5t | None | None | None | |
| 2 | Valspar Paint and Primer in one | 1t | None | None | 1 | CFU |
| | | 2t | None | None | None | |
| | | 3t | None | None | None | |
| | | 4t | None | None | None | |
| | | 5t | None | None | None | |
| 3 | Sherwin-Williams Extreme | 1t | None | None | 1 | CFU |
| | Block Interior/Exterior | 2t | None | None | None | |
| | StainBlocking Primer | 3t | None | None | None | |
| | | 4t | None | None | None | |
| | | 5t | None | None | None | |
| 4 | Sherwin-Williams PVA Drywall | 1t | None | None | 1 | CFU |
| | Primer & Sealer | 2t | None | None | None | |
| | | 3t | None | None | None | |
| | | 4t | None | None | None | |
| | | 5t | None | None | None | |
| 5 | Sherwin-Williams Extreme | 1t | None | None | None | |
| | Bond Primer | 2t | None | None | None | |
| | | 3t | None | None | None | |
| | | 4t | None | None | None | |
| | | 5t | None | None | None | |
| 6 | Foster Products Full Defense | 1t | None | None | None | |
| | 40-20 Fungicidal Coating | 2t | None | None* | None | |
| | | 3t | None | None | None | |
| | | 4t | None | None | None | |
| | | 5t | None | None | None | |
| 7 | Zinsser Mold Killing Primer | 1t | None | Growth | 15 | CFU * |
| | | 2t | None | Growth | 14 | CFU * |
| | | 3t | None | Growth | 2 | CFU * |
| | | 4t | None | Growth | 12 | CFU |
| | | 5t | None | Growth | 22 | CFU |

| | | | | | |
|---|---|---|---|---|---|
| 8 | Kilz Mold + Mildew Primer | 1t | None | None | None |
| | | 2t | None | None | None |
| | | 3t | None | None | None |
| | | 4t | None | None | None |
| | | 5t | None | None | None |
| 9 | Fiberlock Technologies | 1t | None | None | None |
| | Aftershock Fungicidal Coating | 2t | None | None | None |
| | | 3t | None | None | None |
| | | 4t | None | None | None |
| | | 5t | None | None | None |
| 10 | Sherwin-Williams Paintshield | 1t | None | None | None |
| | Interior Latex Paint, Eggshell | 2t | None | None | None |
| | | 3t | None | None | None |
| | | 4t | None | None | None |
| | | 5t | None | None | None |
| 11 | Sherwin-Williams Loxon XP, | 1t | None | None | None |
| | Flat Extra White | 2t | None | None | None |
| | | 3t | None | None | None |
| | | 4t | None | None | None |
| | | 5t | None | None | None |
| 12 | Sherwin-Williams Duration | 1t | None | None | None |
| | Exterior Paint, Flat, Extra | 2t | None | None | None |
| | White | 3t | None | None | None |
| | | 4t | None | None | None |
| | | 5t | None | None | None |
| 13 | Sherwin-Williams Resilience | 1t | None | None | None |
| | Exterior Paint, Satin, Extra | 2t | None | None | None |
| | White | 3t | None | None | None |
| | | 4t | None | None | None |
| | | 5t | None | None | None |

* Indicates recovery of organism other than test organism (Trichophyton interdigitale, previously Trichophyton mentagrophytes, ATCC 9533).

Based on the results of the testing completed, the method of the present disclosure was able to show differentiation for the samples submitted. The drawdown application demonstrated 3 slight failures based on 1 CFU out of 5 replicates of the test organism recovered from the spread plate of the primary neutralization vessel and one clear failure (sample 7, Zinsser Mold Killing Primer) based on growth in the secondary neutralization vessel and spread plates from the primary neutralization vessel.

The spray application demonstrated two slight failures with 1 CFU out of five replicates based on growth in the secondary neutralization vessel and spread plates from the primary neutralization vessel. All samples from the spray application demonstrated contamination on the spread plates from the primary neutralization vessels, possibly due to the sprayer not being able to be sanitized. Nonetheless, the method of the present disclosure was able to differentiate between samples showing growth of the test organism from those not showing test organism growth.

Equivalent results of the two application methods were only demonstrated for three of the five samples demonstrating failure, suggesting a difference in microbicidal quality based on means of application (spray vs. drawdown). It is postulated that the highly variable film thickness applied by the sprayer due to the different viscosities of the sample may have contributed to the disparity in results.

The foregoing examples demonstrate that methods of the present disclosure are able to assess the microbicidal characteristics of a viscous liquid composition, such as a coating, applied by means that require direct contact between the coating composition and applicator.

What has been described above includes examples of the present specification. It is, of course, not possible to describe every conceivable combination of components or methodologies for purposes of describing the present specification, but one of ordinary skill in the art may recognize that many further combinations and permutations of the present specification are possible. Each of the systems, components, and/or methodologies described above may be combined or added together in any permutation. Accordingly, the present specification is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

### LIST OF EMBODIMENTS

Embodiment 1: A method for assessing the microbicidal characteristics of a viscous liquid comprising: a) Forming a microbial film on one or more carriers; b) Exposing the microbially-treated carriers to a uniform film of a viscous liquid while securing the carriers on a platform containing one or more wells; c) Lifting the film-coated carriers from the one or more wells of the platform using a lifting tool to lift the carriers out of the one or more wells; d) Removing the film-coated carriers from the platform using a removal tool; e) Incubating the film-coated carriers in media; and f) Determining the viability of microbes on the microbial-treated carriers to assess the microbicidal characteristic of the viscous liquid.
Embodiment 2: The method of Embodiment 1 wherein the uniform film is formed by smearing, rolling, brushing, or drawing down the viscous liquid on the microbial-treated carrier.
Embodiment 3: The method of Embodiment 1 wherein the uniform film is prepared by a drawdown process using a bar to uniformly apply the viscous liquid on the microbial treated carrier.
Embodiment 4: The method of any one of Embodiments 1 to 3, wherein the platform includes a single well.
Embodiment 5: The method of any one of Embodiments 1 to 3, wherein the platform includes two or more wells.
Embodiment 6: The method of any one of Embodiments 1 to 5, wherein at least one carrier is a glass slide.
Embodiment 7: The method of any one of Embodiments 1 to 5, wherein at least one carrier is metal.
Embodiment 8: The method any one of Embodiments 1 to 5, wherein at least one carrier includes a polymer.
Embodiment 9: The method of any one of the preceding Embodiments, wherein the carriers are sterile prior to forming the microbial film.
Embodiment 10: The method of any one of the preceding Embodiments, wherein the viscous liquid is a paint or coating.
Embodiment 11: The method of any one of the preceding Embodiments, wherein incubation of the film-coated carrier is for a predetermined incubation time at a temperature greater than ambient temperature.
Embodiment 12: The method of any one of the preceding Embodiments, wherein the media after the predetermined incubation time is further neutralized with a neutralizing reagent.
Embodiment 13: The method of any one of the preceding Embodiments, wherein the determination of viability is a qualitative or quantified measurement.
Embodiment 14: The method of any one of Embodiments 1 to 12, wherein the determination of viability is a qualitative measurement of turbidity.
Embodiment 15: The method of any one of Embodiments 1 to 12 wherein the determination of viability is a qualitative measurement of the turbidity of a solution after neutralization.
Embodiment 16: The method of any one of Embodiments 1 to 12 wherein the determination of viability is a quantitative measurement of a spread place of a solution after neutralization.
Embodiment 17: The method of any preceding Embodiment wherein the determination step assesses the microbicidity of the viscous liquid against the microbial film.
Embodiment 18: A method for depositing a thin film of a liquid for testing on a treated carrier, comprising: a) Forming a microbial film on one or more carriers; b) Exposing the carriers treated with the microbial film to a thin film of a liquid while securing the carriers on a platform containing one or more wells, the thin film deposited by drawing down the liquid on the platform; c) Lifting the film-coated carriers from the wells using a lifting tool; and d) Removing the film-coated carriers from the platform using a removal tool.
Embodiment 19: The method of any preceding Embodiment, wherein the microbial film comprises fungal spores.
Embodiment 20: The method of any preceding Embodiment, wherein the microbial film comprises a mold.
Embodiment 21: The method of any preceding Embodiment, wherein the microbial film comprises a bacteria.
Embodiment 22: The method of any preceding Embodiment, wherein the microbial film comprises a virus.
Embodiment 23: The method of any preceding Embodiment, wherein the microbial film comprises a cell.
Embodiment 24: The method of any preceding Embodiment, wherein the viscous liquid comprises a medicinally active compound.
Embodiment 25: A method for assessing the resistance of a paint or coating to mold or fungal growth comprising: a) Preparing microbial-treated carriers; b) Placing the microbial-treated carriers into a platform containing one or more wells; c) Forming a uniform film of the paint or coating on the microbial-treated carriers using a drawdown bar and the platform containing multiple wells to expose the microbial-treated coated carriers to the paint or coating; d) Lifting the film-coated carriers from the one or more wells using a lifting tool; e) Removing the film-coated carriers from the one or more wells using a removal tool; f) Incubating the film-coated carriers in media for a predetermined incubation time at a temperature greater than ambient temperature; g) Neutralizing the media with a neutralizing reagent solution; and h) Determining the viability of microbes in the neutralization reagent solution to assess the resistance of the paint or coating to mold or fungal growth by a qualitative measurement of the turbidity of the neutralizing solution.
Embodiment 26: A system to prepare uniform films on a carrier comprising: a platform containing one or more wells to hold the carrier, a lifting tool to lift a microbial-coated carrier from the wells in the platform, and a removal tool to move a carrier from the platform.
Embodiment 27: The system of Embodiment 26, wherein the wells include apertures for receiving the lifting tool.
Embodiment 28: The system of Embodiment 27 wherein the removal tool comprises prongs to move the carrier from the wells in the platform.
Embodiment 29: A test kit for assessing the resistance of a paint or coating to mold or fungal growth comprising carriers, a platform including one or more wells, a lifting tool to lift one or more microbial-coated carriers from a well in the platform, and a removal tool to move the microbial-coated carriers from the multi-well platform.
Embodiment 30: The test kit of Embodiment 29 further comprising microbes, and growth media reagents.
Embodiment 31: The test kit of any one of Embodiments 28 and 29, further comprising a drawdown bar.
Embodiment 32: The test kit of any one of Embodiments 28 to 30 further comprising a neutralization reagent.
Embodiment 33: The test kit of any one of Embodiments 28 to 31 further comprising incubating and neutralizing vessels.
Embodiment 34: The test kit of any one of Embodiments 28 to 32 wherein the microbes are mold or fungal spores.
Embodiment 35: A method of assessing the resistance of a paint sample to mold of fungal growth comprising using the test kit of any preceding claim.
Embodiment 36: A method of assessing the microbicidal properties of a paint sample comprising using the test kit of any preceding claim.

Illustrative embodiments have been described, hereinabove. It will be apparent to those skilled in the art that the above compositions and methods may incorporate changes and modifications without departing from the general scope of this disclosure. The general scope of the disclosure is intended to encompass all such modifications and alterations.

1. A method for assessing the microbicidal characteristics of a viscous liquid comprising
a. Forming a microbial film on one or more carriers;
b. Exposing the microbially-treated carriers to a uniform film of a viscous liquid while securing the carriers on a platform containing one or more wells;
c. Lifting the film-coated carriers from the one or more wells of the platform using a lifting tool to lift the carriers out of the one or more wells;
d. Removing the film-coated carriers from the platform using a removal tool;
e. Incubating the film-coated carriers in media; and
f. Determining the viability of microbes on the microbial-treated carriers to assess the microbicidal characteristic of the viscous liquid.

2. The method of claim 1 wherein the uniform film is formed by smearing, rolling, brushing, or drawing down the viscous liquid on the microbial-treated carrier.

3. The method of claim 1 wherein the uniform film is prepared by a drawdown process using a bar to uniformly apply the viscous liquid on the microbial treated carrier.

4. The method of any one of claims 1 to 3, wherein the platform includes a single well.

5. The method of any one of claims 1 to 3, wherein the platform includes two or more wells.

6. The method of any one of claims 1 to 5, wherein at least one carrier is a glass slide.

7. The method of any one of claims 1 to 5, wherein at least one carrier is metal.

8. The method any one of claims 1 to 5, wherein at least one carrier includes a polymer.

9. The method of any preceding claim, wherein the carriers are sterile prior to forming the microbial film.

10. The method of any preceding claim, wherein the viscous liquid is a paint or coating.

11. The method of any preceding claim wherein incubation of the film-coated carrier is for a predetermined incubation time at a temperature greater than ambient temperature.

12. The method of any preceding claim wherein the media after the predetermined incubation time is further neutralized with a neutralizing reagent.

13. The method of any preceding claim wherein the determination of viability is a qualitative or quantified measurement.

14. The method of any one of claims 1 to 12 wherein the determination of viability is a qualitative measurement of turbidity.

15. The method of any one of claims 1 to 12 wherein the determination of viability is a qualitative measurement of the turbidity of a solution after neutralization.

16. The method of any preceding claim wherein the determination step assesses the microbicidity of the viscous liquid against the microbial film.

17. A method for depositing a thin film of a liquid for testing on a treated carrier, comprising:
a. Forming a microbial film on one or more carriers;
b. Exposing the carriers treated with the microbial film to a thin film of a liquid while securing the carriers on a platform containing one or more wells, the thin film deposited by drawing down the liquid on the platform;
c. Lifting the film-coated carriers from the wells using a lifting tool; and
d. Removing the film-coated carriers from the platform using a removal tool.

18. The method of any preceding claim, wherein the microbial film comprises fungal spores.

19. The method of any preceding claim, wherein the microbial film comprises a mold.

20. The method of any preceding claim, wherein the microbial film comprises a bacteria.

21. The method of any preceding claim, wherein the microbial film comprises a virus.

22. The method of any preceding claim, wherein the microbial film comprises a cell.

23. The method of any preceding claim, wherein the viscous liquid comprises a medicinally active compound.

24. A method for assessing the resistance of a paint or coating to mold or fungal growth comprising:
a. Preparing microbial-treated carriers;
b. Placing the microbial-treated carriers into a platform containing one or more wells;
c. Forming a uniform film of the paint or coating on the microbial-treated carriers using a drawdown bar and the platform containing multiple wells to expose the microbial-treated coated carriers to the paint or coating;
d. Lifting the film-coated carriers from the one or more wells using a lifting tool; and
e. Removing the film-coated carriers from the one or more wells using a removal tool;
f. Incubating the film-coated carriers in media for a predetermined incubation time at a temperature greater than ambient temperature;
g. Neutralizing the media with a neutralizing reagent solution; and
h. Determining the viability of microbes in the neutralization reagent solution to assess the resistance of the paint or coating to mold or fungal growth by a qualitative measurement of the turbidity of the neutralizing solution.

25. A system to prepare uniform films on a carrier comprising: a platform containing one or more wells to hold the carrier, a lifting tool to lift a microbial-coated carrier from the wells in the platform, and a removal tool to move a carrier from the platform.

26. The system of claim 25, wherein the wells include apertures for receiving the lifting tool.

27. The system of claim 25 wherein the removal tool comprises prongs to move the carrier from the wells in the platform.

28. A test kit for assessing the resistance of a paint or coating to mold or fungal growth comprising carriers, a platform including one or more wells, a lifting tool to lift one or more microbial-coated carriers from a well in the platform, and a removal tool to move the microbial-coated carriers from the multi-well platform.

29. The test kit of claim 28 further comprising microbes, and growth media reagents.

30. The test kit of any one of claims 28 and 29, further comprising a drawdown bar.

31. The test kit of any one of claims 28 to 30 further comprising a neutralization reagent.

32. The test kit of any one of claims 28 to 31 further comprising incubating and neutralizing vessels.

33. The test kit of any one of claims 28 to 32 wherein the microbes are mold or fungal spores.

34. A method of assessing the resistance of a paint sample to mold of fungal growth comprising using the test kit of any preceding claim.

35. A method of assessing the microbicidal properties of a paint sample comprising using the test kit of any preceding claim.
